(19) United States
(12) Patent Application Publication
(10) Pub. No.: US 2026/0355137 A1
(43) Pub. Date: Nov. 12, 2026

(54) NOVEL OPHTHALMIC FORMULATION

(71) Applicant: FERTIS INDIA PVT LTD, Hyderabad, Telangana (IN)

(72) Inventors: Rahul Raju KANUMURU, Hyderabad (IN); Shaji George KOCHUMALAYIL, Hyderabad (IN); Ravikumar SURANENI, Hyderabad (IN)

(73) Assignee: FERTIS INDIA PVT LTD, Hyderabad, Telangana (IN)

(21) Appl. No.: 19/130,842

(22) PCT Filed: Nov. 20, 2023
(86) PCT No.: PCT/IN2023/051069

§ 371 Date: May 16, 2025

(30) Foreign Application Priority Data
Nov. 18, 2022 (IN) 202241066170

(51) Int. Cl. A61K 31/192; A61K 9/00; A61K 31/165; A61K 31/216; A61K 31/352; A61K 47/02; A61K 47/22; A61K 47/34; A61K 47/38; A61K 47/40; A61K 47/42; A61P 27/06; A61P 27/12

(52) U.S. Cl. CPC A61K 31/192; A61K 9/0048; A61K 31/165; A61K 31/216; A61K 31/352; A61K 47/02; A61K 47/22; A61K 47/34; A61K 47/38; A61K 47/40; A61K 47/42; A61P 27/06; A61P 27/12

(57) ABSTRACT

The present invention discloses an ophthalmic formulation/composition comprising cinnamic acids or their derivatives, catechins or its derivatives, either alone or in combination thereof together with acceptable excipients for treating/clearing/reducing conditions associated with cataract, intraocular pressure and glaucoma, lenticular degeneration, corneal epithelial proliferation and liquefaction and inflammation.

### TECHNICAL FIELD

The present invention relates to an ophthalmic formulation/composition comprising cinnamic acids or their derivatives, catechins or its derivatives, either alone or in combination thereof together with acceptable excipients for treating/clearing/reducing conditions associated with cataractous lens, intraocular pressure, glaucoma, lenticular degeneration, corneal epithelial proliferation/liquefaction/inflammation.

### BACKGROUND OF THE INVENTION

A cataract is defined as any opacity in the ocular lens caused by various etiological factors, which is the major cause of blindness. It impairs seriously vision and quality of life.

Glaucoma is a group of eye conditions that damage the optic nerve, which is essential for vision. This damage is often caused by abnormally high pressure in the eye, known as intraocular pressure (IOP). Glaucoma is a leading cause of irreversible blindness worldwide.

Cinnamic acid derivatives and some catechins exhibit anti-oxidative and anti-inflammatory activities and inhibit several important steps of angiogenesis including proliferation, migration, and adhesion.

MARINA CHEMEROVSKI-GLIKMAN1 et al., published an article in Nature titled “Rosmarinic Acid Restores Complete Transparency of Sonicated Human Cataract Ex Vivo and Delays Cataract Formation In Vivo”, Jun. 2018, p. 6-7, wherein, the study rats were treated with a subcutaneous injection of Rosmarinic acid. The article discloses a subcutaneous injection of Rosmarinic acid at a high concentration (40 mM, 500 μl that is 1.4% concentration with each injection dose of 7 mg/0.5 ml) in rats to treat cataracts.

PCT/EP94/03340 discloses that N-acetyl carnosine is a pro-drug for L-carnosine, and proposes a topical medicant containing N-acetyl carnosine useful in the prevention and therapy of cataract.

N-acetyl carnosine drops are routinely administered for the treatment of age-related cataracts.

U.S. Pat. No. 6,194,457 discloses a liquid eye composition comprising an isotonic solution of reduced glutathione, vitamin A, vitamin E, and one or more buffering agents, said buffering agents producing a pH in said composition similar to mammalian eye fluids, wherein said reduced glutathione comprises glutamic acid, cysteine and glycine.

The present inventors felt that there remains a need in the art to provide an efficient ophthalmic formulation that increases the bioavailability of cinnamic acids or their derivatives, catechins or its derivatives, repair the cataractous crystalline lens protein, reduce the intraocular pressure and thereby preventing the occurrence of glaucoma.

### Objective of the Invention

Therefore, it is an objective of the present invention to provide an ophthalmic formulation comprising cinnamic acids or their derivatives, catechins or its derivatives, either alone or in combination thereof together with acceptable excipients effective to increase intraocular absorption of the active ingredients thereby treating/clearing/reducing the cataractous crystalline lens protein.

The further objective is to provide the ophthalmic formulation that treating/clearing/reducing the intraocular pressure and thereby treating glaucoma.

Yet another objective of the present invention is to provide the intraocular formulation that holds promise in preserving vision and enhancing the quality of life for individuals with glaucoma.

Yet another objective of the present invention is to provide a process for the preparation of ophthalmic formulation that holds promise in preserving vision and enhancing the quality of life for individuals with glaucoma.

### SUMMARY OF THE INVENTION

In line with the above objective, the present invention provides an ophthalmic formulation/composition comprising cinnamic acids or its derivatives, catechins or its derivatives, alone or in combination; along with acceptable excipients.

Accordingly, the present invention provides an ophthalmic formulation comprising;
i. Cinnamic acids or its derivatives as active ingredients at a concentration ranging between 0.01% to 5.0% w/w; and/or
ii. Catechins or their derivatives as active ingredients at a concentration ranging between 0.01% to 5.0% w/w; Along with acceptable excipients.

In an aspect the formulation is either powder, liquid or gel.

In an aspect, the cinnamic acids are selected from cinnamic acid, coumaric acid, caffeic acid, rosmarinic acid, ferulic acid and the like.

In an aspect, the derivatives of cinnamic acid esters are selected from alkyl/aralkyl alcohols esterified with appropriate cinnamic acids having general formula;

CA-CO—OR (I)
wherein CA=cinnamic acid and R═C1-C22 alkyl, aryl, heteroaryl,
Ar—CH₂—CH₂—, carbohydrates, lipids, and CA-CO—O(CH)nO—CO-CA wherein n=2-10

The derivatives of cinnamic acid esters, are selected from the group consisting of ethyl cinnamate, ethyl p-methoxy cinnamate, butyl cinnamate, amyl cinnamate, benzyl cinnamate, phenethyl cinnamate, tyrosol cinnamate, hydroxy tyrosol cinnamate, isobutyl cinnamate, cinnamyl cinnamate, phenethyl coumarate, tyrosol coumarate, hydroxy tyrosol coumarate, n-hexadecyl coumarate, n-octadecyl coumarate, n-docosyl coumarate, phenethyl ferulate, tyrosol ferulate, hydroxy tyrosol ferulate, n-hexadecyl ferulate, n-octadecyl ferulate, n-docosyl ferulate, caffeic acid phenylethyl ester (CAPE), tyrosol caffeate, hydroxy tyrosol caffeate, n-hexadecyl caffeate, n-octadecyl caffeate, n-docosyl caffeate, rosmarinic acid, dimeric esters of cinnamic acid, coumaric acid, ferulic acid, caffeic acid where dimeric esters are derived from C₂ to C₁₀ diol and the like.

In another aspect, the derivatives of cinnamic acid amides are selected from amides having general formula;

CA-CO—NHR (II)
wherein CA=cinnamic acid and R is C1-C22 alkyl, aryl, heteroaryl, Aralkyl, lipids and carbohydrates.

The derivatives of cinnamic acid amides, are selected from the group consisting of cinnamoyl phenethyl amine, cinnamoyl tyramine, cinnamoyl serotonin, cinnamoyl dopamine, coumaroyl phenethyl amine, coumaroyl tyramine, coumaroyl dopamine, coumaroyl serotonin, feruloyl phenethyl amine, feruloyl tyramine, feruloyl serotonin, feruloyl dopamine, caffeic acid phenylethyl amide (CAPA), caffeoyl tyramine, caffeoyl serotonin, caffeoyl dopamine and the like.

In a preferred aspect, the cinnamic acid derivatives are selected from cinnamic acid, caffeic acid, rosmarinic acid, ferulic acid, caffeic acid phenylethyl amide (CAPA), caffeic acid phenylethyl ester (CAPE), feruloyl tyramine, caffeoyl tyramine, caffeoyl dopamine, feruloyl dopamine alone or mixtures thereof

In yet another aspect, the catechins or their derivatives are selected from the group consisting of (a) catechin: (2R,3S)-(+)-catechin; (2R,3S)-2-(3,4-dihydroxyphenyl)-3,4-dihydro-2H-chromene-3,5,7-triol; (b) EC: (2R,3S)-(−)-epicatechin; 2-(3,4-Dihydroxyphenyl)-3,4-dihydro-2H-1-benzopyran-3,5,7-triol; (c) ECG: (2R,3R)-(−)-epicatechin gallate; (2R,3R)-2-(3,4-dihydroxyphenyl)-3,4-dihydro-1(2H)-benzopyran-3,5,7-triol 3-(3,4,5-trihydroxy benzoate); (d) EGC: (2R,3R)-(−)-epigallocatechin; (2R,3R)-2-(3,4,5-trihydroxy phenyl)-3,4-dihydro-1(2H)-benzopyran-3,5,7-triol;(e) GC: (2S,3R-(−)-gallocatechin; (2S,3R)-2-(3,4,5-trihydroxy phenyl)-3,4-dihydro-1(2H)-benzo pyran-3,5,7-triol; (f) EGCG: (2R,3R)-(−)-epigallocatechin gallate; (2R,3R)-2-(3,4,5-trihydroxyphenyl)-3,4-dihydro-1(2H)-benzopyran-3,5,7-triol 3-(3,4,5-trihydroxy benzoate); (g) GCG: (2S,3R)-(−)-gallocatechin gallate; (2S,3R)-2-(3,4,5-trihydroxyphenyl)-3,4-dihydro-1(2H)-benzopyran-3,5,7-triol 3-(3,4,5-tri hydroxybenzoate).

In an aspect, the active ingredients in the ophthalmic formulation of the present invention may be encapsulated or in free form.

In another aspect, the acceptable excipients are selected from antioxidants, vitamins, stability enhancers/preservatives, solubility enhancers, buffering agents, isotonic pH adjusters, viscosity enhancers or modifiers, diluents and the like in suitable proportions, with or without antioxidants and vitamins in appropriate proportion.

Accordingly, the stability enhancers/preservatives are selected from 2-Hydroxypropyl β-Cyclodextrin, Polysorbate surfactants including Polysorbate 80 (Tween 80) and Polysorbate 20 (Tween 20); Ethanol, Benzyl alcohol, Propylene glycol, Polyvinylpyrrolidone (PVP), ethylenediaminetetraacetic acid (EDTA); Buffering agents selected from phosphate and citrate buffers, ascorbic acid or sodium metabisulfite; viscosity enhancers or modifiers selected from Carboxymethyl Cellulose (CMC) or its acceptable salt, Hyaluronic acid or its acceptable salt, Hydroxypropyl Methylcellulose (HPMC), Polyvinyl Alcohol (PVA), Glycerin, Polyethylene Glycol (PEG), Xanthan Gum, Guar Gum, Gellan gum, Carbomers like Polyacrylic Acid; Borate buffers such as potassium borate and potassium carbonate; phosphate buffers including sodium phosphate and potassium phosphate, Citrate buffers like sodium citrate or citric acid; Acetate buffers such as sodium acetate, Tris Buffer like Tris(tris(hydroxymethyl)aminomethane; HEPES Buffer like (4-(2-hydroxyethyl)-1-piperazineethanesulfonic acid; antioxidants selected from glutathione, N-acetyl carnosine, vitamins selected from vitamin A, vitamin E. The buffers help to maintain a pH range suitable for eye comfort and stability of the active ingredients.

In the preferred aspect, the acceptable excipients comprise the viscosity enhancer which is carboxymethyl cellulose or an acceptable salt, in an amount ranging from 0.05% to 3.0% w/w, preferably in the range of 0.2% to 2.0% w/w; or hyaluronic acid or an acceptable salt ranging from 0.05% to 1.5% w/w, preferably in the range of 0.05 to 0.3% w/w; 2-hydroxypropyl β-cyclodextrin as encapsulating agent or as a stabilizer in an amount ranging from 0.1% to 15% w/w, preferably 1 to 5% w/w, glutathione or N-acetyl carnosine (NAC) as an antioxidant in an amount of 0.1 to 5% w/w; surfactant selected from tween 80 in an amount ranging from 1-10% preferably 3-6% w/w and vitamins in suitable amounts. based on the total weight of the composition;

In another aspect, the present invention provides an ophthalmic formulation comprises cinnamic acid derivatives in combination with sodium hyaluronate, 2-hydroxypropyl-β-cyclodextrin, carboxymethyl cellulose or an acceptable salt, potassium carbonate and potassium borate buffer optionally with N-acetyl carnosine or glutathione and vitamins in suitable amounts.

In another aspect, the present invention provides an ophthalmic formulation comprising rosmarinic acid with 2-hydroxypropyl β-cyclodextrin combined, sodium hyaluronate, carboxymethyl cellulose or a suitable salt, potassium carbonate, and potassium borate buffer, optionally with N-acetyl carnosine or glutathione and vitamins in suitable amounts.

In yet another aspect, the present invention provides an ophthalmic formulation comprising caffeic acid in combination with 2-hydroxypropyl-β-cyclodextrin combined, hyaluronic acid or a suitable salt, carboxymethyl cellulose or a suitable salt, potassium carbonate and potassium borate buffer, optionally with N-acetyl carnosine or glutathione and vitamins in suitable amounts.

In yet another aspect, the present invention provides an ophthalmic formulation comprising of caffeic acid phenylethyl amide (CAPA) combination with 2-hydroxypropyl-β-cyclodextrin, sodium hyaluronate, carboxymethyl cellulose or a suitable salt, potassium carbonate and potassium borate buffer, optionally with N-acetyl carnosine or glutathione and vitamins in suitable amounts.

In yet another aspect, the present invention provides an ophthalmic formulation comprising of caffeic acid phenylethyl ester (CAPE) in combination with 2-hydroxypropyl-β-cyclodextrin, hyaluronic acid or a suitable salt, carboxymethyl cellulose or a suitable salt, potassium carbonate and potassium borate buffer, optionally with N-acetyl carnosine or glutathione and vitamins in suitable amounts.

In another aspect, the present invention provides an ophthalmic formulation comprising of caffeoyl tyramine in combination with 2-hydroxypropyl-β-cyclodextrin, hyaluronic acid or a suitable salt, carboxymethyl cellulose or a suitable salt, potassium carbonate and potassium borate buffer, optionally with N-acetyl carnosine or glutathione and vitamins in suitable amounts.

In yet another aspect, the present invention provides an ophthalmic formulation comprising of ferulic acid in combination with 2-hydroxypropyl-β-cyclodextrin, hyaluronic acid or a suitable salt, carboxymethyl cellulose or an acceptable salt, potassium carbonate and potassium borate buffer optionally with N-acetyl carnosine or glutathione and vitamins in suitable amounts.

In another aspect, the present invention provides an ophthalmic formulation comprising of feruloyl tyramine in combination with 2-hydroxypropyl-β-cyclodextrin, hyaluronic acid or a suitable salt, carboxymethyl cellulose or a suitable salt, potassium carbonate, and potassium borate buffer, optionally with N-acetyl carnosine or glutathione and vitamins in suitable amounts.

In yet another aspect, the present invention provides an ophthalmic formulation comprising of Epigallocatechin gallate (EGCG), in combination with 2-hydroxypropyl-β-cyclodextrin, hyaluronic acid or a suitable salt, carboxymethyl cellulose or a suitable salt, potassium carbonate, and potassium borate buffer, optionally with N-acetyl carnosine or glutathione and vitamins in suitable amounts.

In another aspect, the present invention provides a process for the preparation of the ophthalmic formulation which comprises the steps of,
i. Dissolving the active ingredients comprising Cinnamic Acids or its derivatives and/or Catechins or its derivatives in sterile water at a temperature ranging between 25-75 °C;
ii. Adding acceptable excipients to the solution of step (i) and stirring the mixture at 25-75 °C;
iii. Adjusting the isotonic pH in the range of 6.2-7.6 with the mixture of base and a buffer; and stirring to a clear and homogenous mixture; and
iv. Packing and storing the ophthalmic grade formulation of step (iii) at 2 to 40 °C.

In an aspect, the active ingredients used in the process for preparation of ophthalmic formulation may be encapsulated or in free form.

### DESCRIPTION OF DRAWINGS

FIG. 1 depicts Representative Images of Histopathology Findings: 1a) Cornea (Uninduced, untreated, Right eye): 1b) Cornea (Induced, untreated, Left eye, G1); 1c) Cornea (Induced, treated, Left eye, G2); 1d) Cornea (Induced, treated, Left eye, G3); 1e) Cornea (Induced, treated, Left eye, G4); 1f) Cornea (Induced, treated, Left eye, G5): 1g) Lens (Uninduced, untreated, Right eye); 1h) Lens (Induced, untreated, Left eye, G1): 1i) Lens (Induced, treated, Left eye, G2); 1j) Lens (Induced, treated, Left eye, G3); 1k) Lens (Induced, treated, Left eye, G4): 1l) Lens (Induced, treated, Left eye G5):

FIG. 2 shows representative Images of Cataract from two animals of each group during induction and treatment: 2a&2b) Un-induced, untreated, right; 2c&2d) Induced, Untreated, G1, left eye; 2e&2f) Induced, treated, G2, left eye; 2g&2h) Induced, treated, G3, left eye; 2i&2j) Induced, treated, G4, left eye; 2k&2l) Induced, treated, G5, left eye.

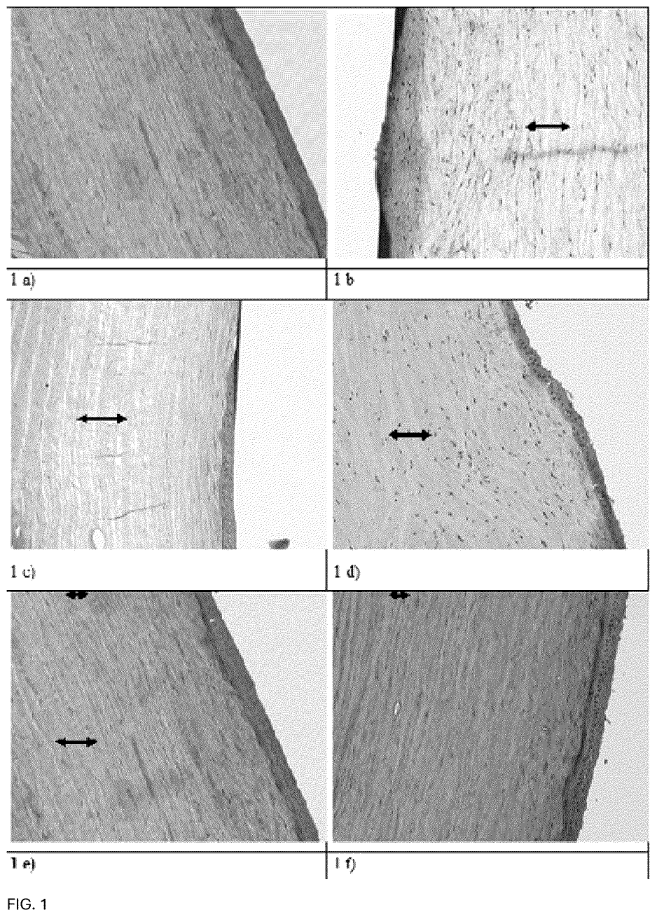

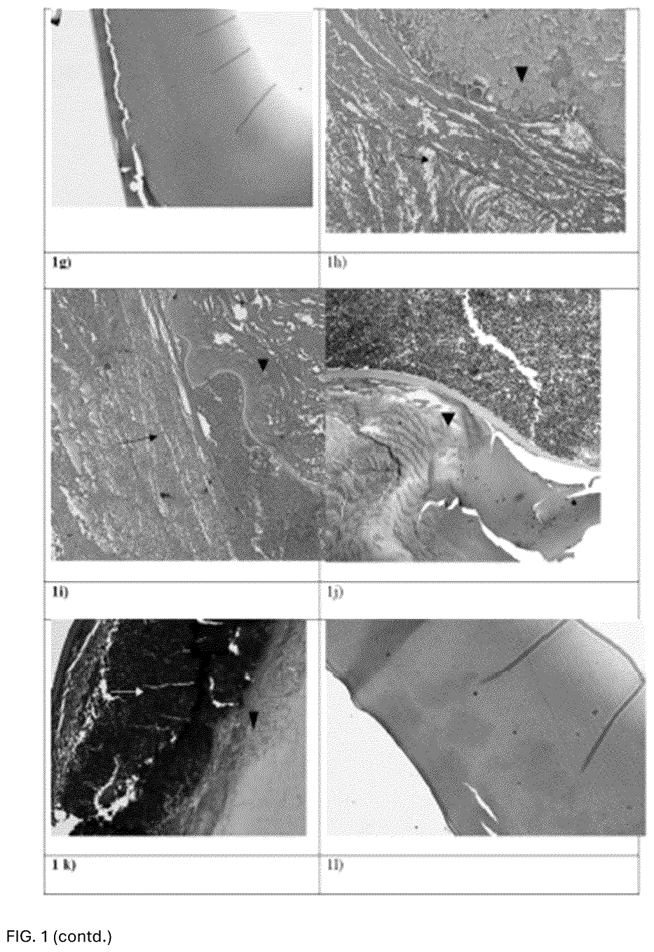

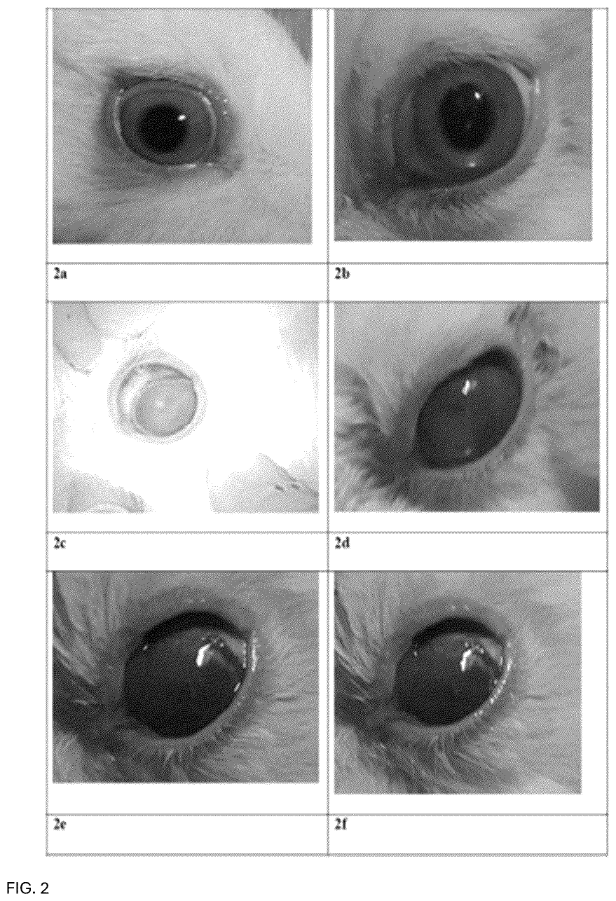

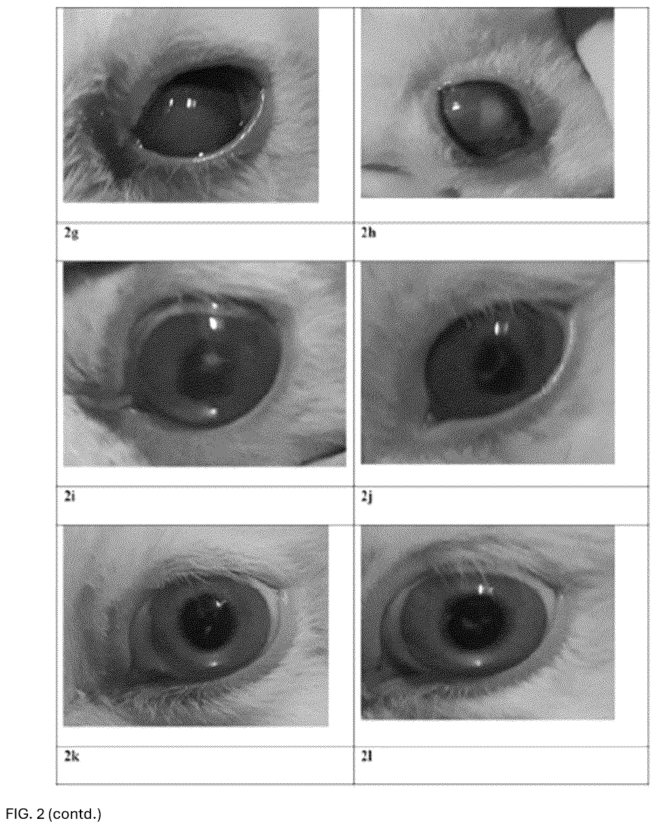

### DETAILED DESCRIPTION OF THE PREFERRED EMBODIMENTS

The invention will now be described in detail in connection with certain preferred and optional embodiments, so that various aspects thereof may be more fully understood and appreciated.

The present invention provides an ophthalmic formulation that can be applied directly on the eyes or as injectable or as oral for treating/clearing/reducing cataract, intraocular pressure and glaucoma. The ophthalmic formulation of the present invention allows the active ingredients to penetrate the corneal and conjunctival epithelium directly, thereby reducing the required dosage and making it a patient-friendly option for treating/clearing/reducing cataracts, intraocular pressure and glaucoma.

In an embodiment, the present invention relates to an ophthalmic formulation comprising;
i. Cinnamic acids or its derivatives as active ingredients; and/or
ii. Catechins or its derivatives as active ingredients.

The active ingredients in the ophthalmic formulation of the present invention are present in an amount ranging between 0.01% to 5.0% w/w.

The ophthalmic formulation of the present invention may further comprise acceptable excipients.

In another embodiment, the present invention relates to an ophthalmic composition/formulation comprising
i. Cinnamic acids or its derivatives as active ingredients at a concentration ranging between 0.01% to 5.0% w/w; and/or
ii. Catechins or their derivatives as active ingredients at a concentration ranging between 0.01% to 5.0% w/w;
   along with acceptable excipients.

In an aspect, the cinnamic acids are selected from cinnamic acid, coumaric acid, caffeic acid, rosmarinic acid, ferulic acid etc

In an embodiment, the ophthalmic formulation of the present invention is a topical oral or injectable.

In an embodiment, the formulation of the present invention is either powder, liquid or gel.

In an embodiment, the derivatives of cinnamic acid esters are selected from alkyl/aralkyl alcohols esterified with appropriate cinnamic acids having general formula

CA-CO—OR (I)
wherein CA=cinnamic acid and R═C1-C22 alkyl, aryl, heteroaryl, Ar—CH₂—CH₂—, carbohydrates, lipids, and CA-CO—O(CH)nO—CO-CA wherein n=2-10

The derivatives of cinnamic acid esters, are selected from the group consisting of ethyl cinnamate, ethyl p-methoxy cinnamate, butyl cinnamate, amyl cinnamate, benzyl cinnamate, phenethyl cinnamate, tyrosol cinnamate, hydroxy tyrosol cinnamate, isobutyl cinnamate, cinnamyl cinnamate, phenethyl coumarate, tyrosol coumarate, hydroxy tyrosol coumarate, n-hexadecyl coumarate, n-octadecyl coumarate, n-docosyl coumarate, phenethyl ferulate, tyrosol ferulate, hydroxy tyrosol ferulate, n-hexadecyl ferulate, n-octadecyl ferulate, n-docosyl ferulate, caffeic acid phenylethyl ester (CAPE), tyrosol caffeate, hydroxy tyrosol caffeate, n-hexadecyl caffeate, n-octadecyl caffeate, n-docosyl caffeate, rosmarinic acid, dimeric esters of cinnamic acid, coumaric acid, ferulic acid, caffeic acid where dimeric esters are derived from C₂ to C₁₀ diol and the like.

In yet another embodiment, the derivatives of cinnamic acid amides are selected from amides having general formula

CA-CO—NHR (II)
wherein
CA=cinnamic acid and R is C1-C22 alkyl, aryl, heteroaryl, Aralkyl, lipids and carbohydrates.

The derivatives of cinnamic acid amides are selected from the group consisting of cinnamoyl phenethyl amine, cinnamoyl tyramine, cinnamoyl serotonin, cinnamoyl dopamine, coumaroyl phenethyl amine, coumaroyl tyramine, coumaroyl dopamine, coumaroyl serotonin, feruloyl phenethyl amine, feruloyl tyramine, feruloyl serotonin, feruloyl dopamine, caffeic acid phenylethyl amide (CAPA), caffeoyl tyramine, caffeoyl serotonin, caffeoyl dopamine and the like.

In a preferred embodiment, the cinnamic acid derivatives as active ingredients are selected from Rosmarinic acid, caffeic acid, ferulic acid, isoferulic acid, homoferulic acid, coumaric acid, 4-methyl-cinnamic acid, 4-amino cinnamic acid, 4-methoxy-cinnamic acid, caffeic acid, 3-hydroxy-4-n-propoxycinnamic acid, sinapic acid, chlorogenic acid, syringic acid, p-coumaric acid and the like.

In yet another embodiment, the catechins or their derivatives are selected from the group consisting of (a) catechin: (2R,3S)-(+)-catechin; (2R,3S)-2-(3,4-dihydroxyphenyl)-3,4-dihydro-2H-chromene-3,5,7-triol; (b) EC: (2R,3S)-(−)-epicatechin; 2-(3,4-Dihydroxyphenyl)-3,4-dihydro-2H-1-benzopyran-3,5,7-triol; (c) ECG: (2R,3R)-(−)-epicatechin gallate; (2R,3R)-2-(3,4-dihydroxyphenyl)-3,4-dihydro-1(2H)-benzopyran-3,5,7-triol 3-(3,4,5-trihydroxy benzoate); (d) EGC: (2R,3R)-(−)-epigallocatechin; (2R,3R)-2-(3,4,5-trihydroxy phenyl)-3,4-dihydro-1(2H)-benzopyran-3,5,7-triol;(e) GC: (2S,3R-(−)-gallocatechin; (2S,3R)-2-(3,4,5-trihydroxy phenyl)-3,4-dihydro-1(2H)-benzopyran-3,5,7-triol; (f) EGCG: (2R,3R)-(−)-epigallocatechin gallate; (2R,3R)-2-(3,4,5-trihydroxyphenyl)-3,4-dihydro-1(2H)-benzopyran-3,5,7-triol 3-(3,4,5-trihydroxy benzoate); (g) GCG: (2S,3R)-(−)-gallocatechin gallate; (2S,3R)-2-(3,4,5-trihydroxyphenyl)-3,4-dihydro-1(2H)-benzopyran-3,5,7-triol 3-(3,4,5-trihydroxy benzoate).

In an embodiment, the active ingredients in the ophthalmic formulation of the present invention may be encapsulated or in free form.

The acceptable excipients are selected from stability enhancers/preservatives, solubility enhancers, buffering agents, isotonic pH adjusters, viscosity enhancers or modifiers, diluents, encapsulating agent, antioxidants, vitamins and the like in suitable proportions.

Accordingly, the stability enhancers/preservatives are selected from 2-Hydroxypropyl-β-Cyclodextrin, Polysorbate surfactants including Polysorbate 80 (Tween 80) and Polysorbate 20 (Tween 20), Ethanol, Benzyl alcohol, Propylene glycol, Polyvinylpyrrolidone (PVP), ethylenediaminetetraacetic acid (EDTA); viscosity enhancers or modifiers selected from Carboxymethyl Cellulose (CMC) or its acceptable salt, Hyaluronic acid or its acceptable salt, Hydroxypropyl Methylcellulose (HPMC), Polyvinyl Alcohol (PVA), Glycerine, Polyethylene Glycol (PEG), Xanthan Gum, Guar Gum, Gellan gum, Carbomers like Polyacrylic Acid; Buffering agents selected from phosphate buffer including sodium phosphate and potassium phosphate; Citrate buffers selected from sodium citrate or citric acid; Borate buffers such as potassium borate; Acetate buffers such as sodium acetate, Tris Buffer like Tris(tris(hydroxymethyl)aminomethane; HEPES Buffer like (4-(2-hydroxyethyl)-1-piperazineethanesulfonic acid; ascorbic acid; sodium metabisulfite; potassium carbonate; antioxidants selected from glutathione and N-acetyl carnosine. The buffers help to maintain an isotonic pH range suitable for eye comfort and stability of the active ingredients.

In the preferred aspect, the acceptable excipients comprise the viscosity enhancer consisting of carboxymethyl cellulose or an acceptable salt, in an amount ranging from 0.05% to 5.0% w/w, preferably in the range of 0.2% to 2.0% w/w; or hyaluronic acid or an acceptable salt ranging from 0.05% to 1.5% w/w, preferably in the range of 0.05 to 0.3% w/w; Glutathione or N-acetyl carnosine (NAC) as an antioxidant in an amount of 0.1 to 5% w/w; 2-hydroxypropyl-β-cyclodextrin as encapsulating agent or as a stabilizer in an amount ranging from 0.1% to 15% w/w, preferably 1 to 5% w/w; surfactant selected from Tween 80 in an amount ranging from 1-10% w/w preferably 3-6% w/w, vitamins 0.5-6% w/w and the like in suitable proportions. based on the total weight of the composition.

In accordance with the present invention, one of the suitable formulations advantageously comprises the following ingredients in the given amounts: Rosmarinic acid 0.5% w/w; Carboxymethylcellulose sodium 0.3% w/w Sodium hyaluronate 0.1% w/w; 2-hydroxypropyl-β-cyclodextrin 1% w/w; and Potassium Borate, Potassium Bicarbonate, or any other suitable buffers necessary to adjust the pH of the ophthalmic formulation to a pH in the range of 6.2 to 7.6, Sterile Water.

In an embodiment, the present invention relates to ophthalmic formulation comprising;
i. 0.5% w/w of Rosmarinic acid.
ii. 0.3% w/w of Carboxymethylcellulose sodium, 0.1% w/w of Sodium hyaluronate; 1% w/w of 2-hydroxypropyl-β-cyclodextrin;
iii. Potassium Borate and Potassium Bicarbonate in appropriate proportion to adjust the pH of the ophthalmic formulation to a pH in the range of 6.2 to 7.6; and
iv. Sterile Water

In another embodiment, the present invention relates to ophthalmic formulation comprising;
i. 0.5% w/w of Caffeic acid;
ii. 0.3% w/w of Carboxymethylcellulose sodium; 0.1% w/w of Sodium hyaluronate; 1% w/w of 2-hydroxypropyl-β-cyclodextrin;
iii. Potassium Borate and Potassium Bicarbonate in appropriate proportion to adjust the pH of the ophthalmic formulation to a pH in the range of 6.2 to 7.6; and
iv. Sterile Water

In a further embodiment, the present invention relates to ophthalmic formulation comprising;
i. 0.5% w/w of encapsulated Feruloyl tyramine,
ii. 0.3% w/w of Carboxymethylcellulose sodium; 0.1% w/w of Sodium hyaluronate; 2.32% w/w of 2-hydroxypropyl-β-cyclodextrin;
iii. Potassium Borate and Potassium Bicarbonate in appropriate proportion to adjust the pH of the ophthalmic formulation to a pH in the range of 6.2 to 7.6;
iv. 6% w/w Tween 80; and
v. Sterile Water.

In another embodiment, the present invention discloses the ophthalmic formulation comprising;
i. 0.5% w/w of Ferulic acid;
ii. 0.3% w/w of Carboxymethylcellulose sodium, 0.1% w/w of Sodium hyaluronate, 1% w/w of 2-hydroxypropyl-β-cyclodextrin;
iii. Potassium Borate and Potassium Bicarbonate in appropriate proportion to adjust the pH of the ophthalmic formulation to a pH in the range of 6.2 to 7.6; and
iv. Sterile Water.

In another embodiment, the present invention relates to ophthalmic formulation comprising;
i. 0.5% w/w of Caffeic Acid Phenylethyl Ester (CAPE),
ii. 0.3% w/w of Carboxymethylcellulose sodium; 0.1% w/w of Sodium hyaluronate; 2.05% w/w of 2-hydroxypropyl-β-cyclodextrin;
iii. Potassium Borate and Potassium Bicarbonate in appropriate proportion to adjust the pH of the ophthalmic formulation to a pH in the range of 6.2 to 7.6; and
iv. Sterile Water

In another embodiment, the present invention relates to ophthalmic formulation comprising;
i. 0.5% w/w of Caffeic Acid Phenylethyl Amide (CAPA);
ii. 0.3% w/w of Carboxymethylcellulose sodium; 0.1% w/w of Sodium hyaluronate; 2.16% w/w of 2-hydroxypropyl-β-cyclodextrin;
iii. Potassium Borate and Potassium Bicarbonate in appropriate proportion to adjust the pH of the ophthalmic formulation to a pH in the range of 6.2 to 7.6; and
iv. Sterile Water

In yet another embodiment, the present invention relates to an ophthalmic formulation comprising;
i. 0.5% w/w of Rosmarinic acid;
ii. 0.25% w/w of Caffeic acid;
iii. 0.3% w/w of Carboxymethylcellulose sodium, 0.1% w/w of Sodium hyaluronate, 1% w/w of 2-hydroxypropyl-β-cyclodextrin;
iv. Potassium Borate and Potassium Bicarbonate in appropriate proportion to adjust the pH of the ophthalmic formulation to a pH in the range of 6.2 to 7.6; and
v. Sterile Water.

In an embodiment, the present invention relates to an ophthalmic formulation comprising;
i. 0.15-0.5% w/w of Rosmarinic acid;
ii. 0.25-0.5% w/w of Caffeic acid;
iii. 0.15-0.5% w/w of Feruloyl tyramine;
iv. 0.3% w/w of Carboxymethylcellulose sodium, 0.1% w/w of Sodium hyaluronate, 1% w/w of 2-hydroxypropyl β-cyclodextrin;
v. Potassium Borate and Potassium Bicarbonate in appropriate proportion to adjust the pH of the ophthalmic formulation to a pH in the range of 6.2 to 7.6;
vi. 6% Tween 80; and
vii. Sterile Water

In another embodiment, the present invention relates to an ophthalmic formulation comprising;
i. 0.15-0.5% w/w of Rosmarinic acid;
ii. 0.15-0.5% w/w of Caffeic acid;
iii. 0.15-0.5% w/w of Feruloyl tyramine;
iv. 0.15-0.5% w/w of Epigalacto catechin gallate;
v. 0.3% w/w of Carboxymethylcellulose sodium, 0.1% w/w of Sodium hyaluronate, 1% w/w of 2-hydroxypropyl β-cyclodextrin;
vi. Potassium Borate and Potassium Bicarbonate in appropriate proportion to adjust the pH of the ophthalmic formulation to a pH in the range of 6.2 to 7.6;
vii. 8% Tween 20; and
viii. Sterile Water.

In an embodiment, the present invention relates to an ophthalmic formulation comprising;
i. 0.25-0.5% w/w of Epigalacto catechin gallate;
ii. 0.3% w/w of Carboxymethylcellulose sodium, 0.1% w/w of Sodium hyaluronate, 1% w/w of 2-hydroxypropyl-β-cyclodextrin;
iii. Potassium Borate and Potassium Bicarbonate in appropriate proportion to adjust the pH of the ophthalmic formulation to a pH in the range of 6.2 to 7.6; and
iv. Sterile Water.

In an embodiment, the ophthalmic composition/formulation of the present invention enhances the absorption of the active ingredients within the eye consequently treating/clearing/reducing the cataractous crystalline protein, intraocular pressure, and Glaucoma.

In an embodiment, the ophthalmic formulation of the present invention comprising Rosmarinic acid is used at a much lower concentration as compared to the composition/formulation known in the art. MARINA CHEMEROVSKI-GLIKMAN1 etal., published an article in Nature titled ‘Rosmarinic Acid Restores Complete Transparency of Sonicated Human Cataract ex vivo and Delays Cataract Formation in vivo”, Jun. 2018, p. 6-7, where the study conducted on rats were provided with a subcutaneous injection of Rosmarinic acid (40 mM, 500 μl that is 1.4% concentration with each injection dose of 7 mg/0.5 ml), to treat cataract, whereas the eye formulation provided in the present invention comprises significantly lower concentration of 0.5% Rosmarinic acid with the dosage of 0.5 mg/100 μl for ocular administration.

In an embodiment, the ophthalmic formulation of the present invention enhances the intraocular absorption of the active ingredients resulting in treating/clearing/reducing cataractous proteins. Additionally, it contributes to reducing intraocular pressure, offering an effective approach for treating/reducing clearing/glaucoma.

In an embodiment, the encapsulation of Rosmarinic acid with 2-hydroxy-β-cyclodextrin improves stability, solubility, and gradual release, reducing intraocular pressure, increasing aqueous humor outflow, and potentially treating glaucoma.

In yet another embodiment, the present invention discloses the general process for the preparation of the ophthalmic formulation comprises the steps of,
i. Dissolving the active ingredients comprising Cinnamic Acids or its derivatives and/or catechins or its derivatives in sterile water at a temperatures ranging between 25-75 °C;
ii. Adding acceptable excipients to the solution of step (i) and stirring the mixture at 25-75 °C;
iii. Adjusting the pH in the range of 6.2 to 7.6 with the mixture of base and a buffer; and stirring to a clear and homogenous mixture; and
iv. Packing and storing the ophthalmic formulation of step (iii) at 2-40 °C.

In an embodiment, the active ingredients may be encapsulated or in the free form.

Accordingly, the present invention provides a process for the preparation of ophthalmic formulation comprising dissolving the active ingredients comprising Cinnamic Acids or its derivatives and/or Catechins or its derivatives in the free form, in 100 mL of sterile water at a temperature range of 25-30 °C while being stirred. Following this, suitable active excipients are added to the solution, and the suspension was stirred at room temperature for a duration of 2 hours. Subsequently, the pH of the resulting suspension was carefully adjusted within the range of 6.2-7.6 using a mixture of Potassium carbonate and Potassium borate buffer. After achieving the desired pH, the solution was stirred continuously for an additional 30 minutes until it attained a clear and homogeneous consistency.

The final sterile formulation of ophthalmic grade was then packed, labelled, and stored under controlled conditions at temperatures ranging from 2-40 °C.

In another embodiment, the present invention discloses a process for the preparation of an ophthalmic formulation comprising adding the Encapsulated active ingredients in 100 mL of sterile water at a controlled temperature of 65-75 °C. Following this, the formulation excipients were added to the solution. The resulting suspension was stirred at 65-75 °C, extending for a duration of 12 hours. The pH of the suspension was then adjusted within the range of 6.2-7.6, employing the mixture of Potassium carbonate and Potassium borate buffer. The pH adjustment is a critical step to ensure the formulation's suitability for ophthalmic use. To aid in the dissolution of encapsulated Feruloyl Tyramine, 6.0 g of Tween-80 was introduced into the sterile water. Subsequently, the solution was stirred for an additional 30 minutes at 65-75 °C until it achieved the desired state of clarity and homogeneity. Upon the successful completion of the procedure the solution was cooled to room temperature, the sterile encapsulated Feruloyl tyramine-based formulation was packed, labelled, and stored at 2-40 °C.

In yet another embodiment, the present invention provides the ophthalmic formulation/composition comprising cinnamic acids or its derivatives; catechins or its derivatives as active ingredients alone or in combination thereof together with acceptable excipients, wherein said formulation enhances the absorption of the active ingredients within the eye thereby aids in treating/clearing/reducing the cataractous affected crystalline proteins, helps in reducing the intraocular pressure and is effective in preventing the glaucoma.

In an embodiment, the present invention discloses the ophthalmic formulation for treating/clearing/reducing: cataractous lens, intraocular pressure, glaucoma, lenticular degeneration, corneal epithelial proliferation/liquefaction/inflammation.

In another embodiment, the present invention relates to a method for treating/clearing/reducing: cataractous lens, intraocular pressure, glaucoma, lenticular degeneration, corneal epithelial proliferation/liquefaction/inflammation and their occurrence/reoccurrence comprising applying/administering the ophthalmic formulation to the subject in need thereof.

The ophthalmic formulations of the present invention comprising the active ingredients selected from cinnamic acids or its derivatives; or catechins or its derivatives; alone or in combination provides the following advantages:
Increase Aqueous Humor Outflow and reducing the intraocular pressure (IOP): By promoting the efficient drainage of aqueous humor, these formulations target the regulation of intraocular pressure (IOP).
Reduce Ocular Inflammation: One of the intended effects is the reduction of inflammation within the eye, particularly in cases associated with elevated IOP.
Provide Neuroprotection to Retinal Ganglion Cells: These formulations are designed to offer neuroprotection to retinal ganglion cells, which is vital for supporting vision and potentially contributing to the IOP reduction results in prevention of glaucoma.
Antioxidant Properties: Included in their mechanism of action is the protection of trabecular meshwork cells from oxidative damage, thereby preserving normal fluid drainage and remove cataract-forming proteins, offering further potential benefits for ocular health.
Reduces Lenticular Degeneration
Reduces epithelial proliferation.
Reduces corneal Liquefaction.

The following example, which includes preferred embodiments, will serve to illustrate the practice of this invention, it being understood that the particulars shown are by way of example and for purpose of illustrative discussion of preferred embodiments of the invention.

### Example 1

The table below illustrates the various ophthalmic formulations of the present invention.

TABLE 1

| | | Percentage | | Percentage | |
|---|---|---|---|---|---|
| S. | Active | (%) of Active | | (%) of | |
| NO | ingredient | ingredient | Excipients | Excipients | |
| 1 | Caffeic acid | 0.5 | Hyaluronic acid sodium salt | 0.1 | |
| | | | Carboxymethyl cellulose sodium | 0.3 | |
| | | | 2-hydroxypropyl β-cyclodextrin | 1 | |
| | | | *Potassium bicarbonate | Variable | |
| | | | *Potassium borate | Variable | |
| | | | Sterile Water (mL) | 100 | mL |
| 2 | Caffeic acid | 0.5 | Hyaluronic acid sodium salt | 0.1 | |
| | phenyl ethyl | | Carboxymethyl cellulose sodium | 0.3 | |
| | amine | | 2-hydroxypropyl β-cyclodextrin | 2.05 | |
| | (CAPE) | | *Potassium bicarbonate | Variable | |
| | | | *Potassium borate | Variable | |
| | | | Sterile Water (mL) | 100 | mL |
| 3 | Caffeic | 0.5 | Hyaluronic acid sodium salt | 0.1 | |
| | Acid | | Carboxymethyl cellulose sodium | 0.3 | |
| | Phenylethyl | | 2-hydroxypropyl β-cyclodextrin | 2.16 | |
| | Amide | | *Potassium bicarbonate | Variable | |
| | (CAPA) | | *Potassium borate | Variable | |
| | | | Sterile Water (mL) | 100 | mL |
| 5 | Caffeoyl | 0.5 | Hyaluronic acid sodium salt | 0.1 | |
| | tyramine | | Carboxymethyl cellulose sodium | 0.3 | |
| | | | 2-hydroxypropyl β-cyclodextrin | 2.04 | |
| | | | *Potassium bicarbonate | Variable | |
| | | | *Potassium borate | Variable | |
| | | | Tween 80 | 6 | g |
| | | | Sterile Water (mL) | 100 | mL |
| 6 | Rosmarinic | 0.5 | N-Acetyl carnosine | 1 | |
| | acid | | Hyaluronic acid sodium salt | 0.1 | |
| | | | Carboxymethyl cellulose sodium | 0.3 | |
| | | | 2-hydroxypropyl β-cyclodextrin | 1 | |
| | | | *Potassium bicarbonate | Variable | |
| | | | *Potassium borate | Variable | |
| | | | Sterile Water (mL) | 100 | mL |
| 7 | Rosmarinic | 0.5 | Hyaluronic acid sodium salt | 0.1 | |
| | acid | | Carboxymethyl cellulose sodium | 0.3 | |
| | | | 2-hydroxypropyl β-cyclodextrin | 1 | |
| | | | *Potassium bicarbonate | Variable | |
| | | | *Potassium borate | Variable | |
| | | | Sterile Water (mL) | 100 | mL |
| 8 | Rosmarinic | 0.5 | Hyaluronic acid sodium salt | 0.1 | |
| | acid | | | | |
| | Caffeic acid | 0.25 | | | |
| | | | Carboxymethyl cellulose sodium | 0.3 | |
| | | | 2-hydroxypropyl β-cyclodextrin | 1 | |
| | | | *Potassium bicarbonate | Variable | |
| | | | *Potassium borate | Variable | |
| | | | Sterile Water (mL) | 100 | mL |
| 9 | Rosmarinic | 0.15-0.5 | Hyaluronic acid sodium salt | 0.1 | |
| | acid | | | | |
| | Caffeic acid | 0.25-0.5 | | | |
| | Feruloyl | 0.15-0.5 | Carboxymethyl cellulose sodium | 0.3 | |
| | tyramine | | | | |
| | | | 2-hydroxypropyl β-cyclodextrin | 1 | |
| | | | *Potassium bicarbonate | Variable | |
| | | | *Potassium borate | Variable | |
| | | | Tween 80 | 6 | g |
| | | | Sterile Water (mL) | 100 | mL |
| 10 | Rosmarinic | 0.15-0.5 | Hyaluronic acid sodium salt | 0.1 | |
| | acid | | | | |
| | Caffeic acid | 0.15-0.5 | | | |
| | Feruloyl | 0.15-0.5 | Carboxymethyl cellulose sodium | 0.3 | |
| | tyramine | | | | |
| | Epigalactocat- | 0.15-0.5 | 2-hydroxypropyl β-cyclodextrin | 1 | |
| | echin | | *Potassium bicarbonate | Variable | |
| | gallate | | Tween 80 | 6 | g |

| *Potassium borate | Variable | |
|---|---|---|
| Sterile Water (mL) | 100 | mL |

| | | | | |
|---|---|---|---|---|
| 11 | Ferulic acid | 0.5 | Hyaluronic acid sodium salt | 0.1 |
| | | | Carboxymethyl cellulose sodium | 0.3 |
| | | | 2-hydroxypropyl β-cyclodextrin | 1 |
| | | | *Potassium bicarbonate | Variable |
| | | | *Potassium borate | Variable |

| | | |
|---|---|---|
| Sterile Water (mL) | 100 | mL |

| | | | | |
|---|---|---|---|---|
| 12 | Feruloyl | 0.5 | Hyaluronic acid sodium salt | 0.1 |
| | tyramine | | Carboxymethyl cellulose sodium | 0.3 |
| | | | 2-hydroxypropyl β-cyclodextrin | 2.32 |
| | | | *Potassium bicarbonate | Variable |
| | | | *Potassium borate | Variable |

| | | |
|---|---|---|
| Tween 80 | 6 | g |
| Sterile Water (mL) | 100 | mL |

| | | | | |
|---|---|---|---|---|
| 13 | Epigalactocat- | 0.5 | Hyaluronic acid sodium salt | 0.1 |
| | echin | | Carboxymethyl cellulose sodium | 0.3 |
| | gallate | | 2-hydroxypropyl β-cyclodextrin | 1.0 |
| | | | *Potassium bicarbonate | Variable |
| | | | *Potassium borate | Variable |

| | | |
|---|---|---|
| Sterile Water (mL) | 100 | mL |

| | | | | |
|---|---|---|---|---|
| 14 | Caffeic acid | 0.5 | Hyaluronic acid sodium salt | 0.1 |
| | | | Glutathione | 0.5 |
| | | | Vit E | 0.5 |
| | | | Carboxymethyl cellulose sodium | 0.3 |
| | | | 2-hydroxypropyl β-cyclodextrin | 1 |
| | | | *Potassium bicarbonate | Variable |
| | | | *Potassium borate | Variable |

| | | |
|---|---|---|
| Sterile Water (mL) | 100 | mL |

### Example 2

### A: General Process for the Preparation of the Ophthalmic Formulation as Disclosed in Table 1.

Dissolving the active ingredients in a suitable amount in 100 mL of sterile water at a temperature range of 25-30 °C while being stirred. Following this, excipients were added in suitable proportion to the solution and the suspension was stirred at room temperature for a duration of 2 hours. Subsequently, the pH of the resulting suspension was carefully adjusted within the range of 6.2-7.6 using a mixture of base and buffer. After achieving the desired pH, the solution was continued to be stirred for an additional 30 minutes until it attained a clear and homogeneous consistency. The final sterile formulation was then packed, labelled, and stored under controlled conditions at temperatures ranging from 2-40 °C.

### B: General Process for the Preparation of an Ophthalmic Formulation Containing the Encapsulated Active Ingredient.

Adding 2-hydroxypropyl-β-cyclodextrin to the active ingredient and grinded to obtain the encapsulated active ingredient. This was followed by adding the Encapsulated active ingredient in 100 mL of sterile water at a controlled temperature of 65-75 °C. Following this, the suitable formulation excipients were added to the solution. The resulting suspension was stirred at 65-75 °C, extending for a duration of 12 hours. The pH of the suspension was then adjusted within the range of 6.2-7.6, employing the suitable mixture of base and buffer. The pH adjustment is a critical step to ensure the formulation's suitability for ophthalmic use. To aid in the dissolution of encapsulated active ingredient, suitable amount of the surfactant was added into the sterile water. Subsequently, the solution was continued to be stirred for an additional 30 minutes at 65-75 °C until it achieved the desired state of clarity and homogeneity. Upon the successful completion of the procedure the solution was cooled to room temperature, the sterile encapsulated active-based formulation was packed, labelled, and stored under carefully controlled conditions, maintaining temperature 2-40 °C.

An illustrative ophthalmic formulation for treatment of eye disease is set forth below: The formulations provided herein below are tested in cataract induced New Zealand rabbits, and the results are discussed herein below.

The following preclinical studies were conducted in rabbit model to test the efficacy of various active ingredients.

### Induction of Cataracts and Increasing Intraocular Pressure Leading to Glaucoma.

Cataract was induced by injecting 0.5% Methyl Cellulose, which is replaced by aqueous humor in the rabbit eye (i.e., 0.2 mL of Aqueous humor was withdrawn and the same 0.2 mL of 0.5% Methyl cellulose was injected as a replacement). Cataract was observed within 3-4 days of 0.5% Methyl cellulose replacement. Cataract induction was performed to left eye and right eye was served as a control.

### Cataract and Glaucoma Induction and Confirmation

Rabbits were observed by ophthalmoscope and slit lamp once in three days for cataract confirmation. Intraocular pressure was measured with a tonometer once in three days for the confirmation of glaucoma.

TABLE 2
Formulation details:

| | F1 | F2 | F3 | F4 | Placebo | |
|---|---|---|---|---|---|---|
| Recipe Details | G2 | G3 | G4 | G5 | G1 | Percentage |
| Rosmarinic | 0.25 | 0.5 | 1.0 | 0.5 | 0 | 0.25%, |
| acid | | | | | | 0.5%, 1% |
| N-Acetyl | 1 | 1 | 1 | 0 | 0 | 1% |
| carnosine | | | | | | |
| Hyaluronic acid | 0.1 | 0.1 | 0.1 | 0.1 | 0.1 | 0.10% |
| Na. salt | | | | | | |
| Carboxy methyl | 0.3 | 0.3 | 0.3 | 0.3 | 0.3 | 0.30% |
| cellulose | | | | | | |
| sodium | | | | | | |
| HP-β-CD | 1 | 1 | 1 | 1 | 1 | 1% |
| (Hydroxypropyl | | | | | | |
| derv of β-CD) | | | | | | |
| *Potassium | Variable | Variable | Variable | Variable | Variable | Variable |
| bicarbonate | | | | | | |
| *Potassium | Variable | Variable | Variable | Variable | Variable | Variable |
| borate | | | | | | |
| Sterile- Water | 100 | 100 | 100 | 100 | 100 | NA |
| (mL) | | | | | | |

Note:
50 μL per left eye was given with a gap of 3-5 minutes again installation 50 μL was same eye.

### Route of Administration and Rationale

Test Item formulations were administered through ocular route. Ocular route has been chosen as it is the intended therapeutic route of administration in humans.

### Dose Administration

All test formulations provided as above were administered topically as a therapeutic treatment. A total of 100 μL/Rabbit of placebo or Rosmarinic acid formulations (F1, F2, F3 and F4) were instilled in the 50 μL/eye (left) in two divided doses for 28 days

### Macroscopic Observations:

The following observations were carried out during the study period.

TABLE 3

| S. | Animal | Rosmarinic | |
|---|---|---|---|
| No | Group | acid % | Inference (Left Eye) |
| 1 | G1 | Placebo | Opacity, cornea, diffuse, moderate |
| 2 | G2 | 0.25 | Opacity, cornea, diffuse, moderate |
| | | | Red discolored, cornea, mild |
| 3 | G3 | 0.5 with | Opacity, cornea, diffuse, moderate |
| | | N-acetyl | |
| | | carnosine | |
| 4 | G4 | 1.0 | No abnormality Detected in 4 animals, and |
| | | | they are completely cured, Two Rabbits |
| | | | minimal dot opacity in cornea was observed |
| 5 | G5 | 0.5 | No abnormality Detected and cataract is |
| | | | completely cured in all 6 rabbits |

Note:
Cataract was induced only in left eye of all animals and right eye is completely normal. The inference given in above table is about cataract induced left eye. Cataract development was monitored in terms of Lenticular degeneration and epithelial proliferation.

### Microscopic Observations (Histopathology):

All surviving animals were euthanized on Day 29 by overdose of Thiopental Sodium. Before scheduled euthanasia, the animals were fasted overnight (water allowed) and terminal body weights were recorded. Animals were subjected to detailed macroscopic observation during necropsy. Both right (untreated/uninduced) and left (treated/induced) eyes were collected and cornea and lens were separated, preserved in Davidson fluid. Cornea and lens selected for histopathological evaluation were processed, embedded and sections were cut at 3-5 μm thickness, Sections were placed on grease-free slides and stained with Hematoxylin and Eosin. A 5-step grading system of minimum, mild, moderate, marked and severe will be used to rank microscopic findings for comparison among groups. Data was compiled based on incidences and severity of changes.

### Main Group/Formulation: G1/Placebo Control (I072/001-I072/006)/Gender: Male

TABLE 4

| Organs/ | Severity of Lesions in Individual Animals | | | | | |
|---|---|---|---|---|---|---|
| Histopathology | I072/001 | I072/002 | I072/003 | I072/004 | I072/005 | I072/006 |
| RIGHT EYE | W | W | W | W | W | W |
| LEFT EYE | | | | | | |
| LENS | | | | | | |
| Liquefaction | 5+ | 5+ | 5+ | 3+ | 3+ | 3+ |
| Epithelial | 5+ | 5+ | 5+ | W | 4+ | 4+ |
| Proliferation | | | | | | |
| CORNEA | | | | | | |
| Corneal edema | 3+ | 3+ | 3+ | 3+ | 3+ | 3+ |

TABLE 5
Main Group/Formulation: G2/F1 (I072/007-I072/012)

| Organs/ | Severity of Lesions in Individual Animals | | | | | |
|---|---|---|---|---|---|---|
| Histopathology | I072/007 | I072/008 | I072/009 | I072/010 | I072/011 | I072/012 |
| RIGHT EYE | W | W | W | W | W | W |
| LEFT EYE | | | | | | |
| LENS | | | | | | |
| Liquefaction | 3+ | 2+ | 2+ | 3+ | 3+ | 4+ |
| Epithelial | 4+ | 1+ | 4+ | 4+ | 4+ | 3+ |
| Proliferation | | | | | | |
| CORNEA | | | | | | |
| Corneal edema | 3+ | 3+ | 2+ | 2+ | 2+ | 3+ |

TABLE 6
Main Group/Formulation: G3/F2 (I072/013-I072/018)

| Organs/ | Severity of Lesions in Individual Animals | | | | | |
|---|---|---|---|---|---|---|
| Histopathology | I072/013 | I072/014 | I072/015 | I072/016 | I072/017 | I072/018 |
| RIGHT EYE | W | W | W | W | W | W |
| LEFT EYE | | | | | | |
| LENS | | | | | | |
| Liquefaction | 3+ | 3+ | 1+ | 4+ | 2+ | 4+ |
| Epithelial | 4+ | 4+ | 3+ | 3+ | 2+ | 4+ |
| Proliferation | | | | | | |
| CORNEA | | | | | | |
| Corneal edema | 3+ | 2+ | 2+ | 2+ | 2+ | 3+ |

TABLE 7
Main Group/Formulation: G4/F3 (I072/019-I072/024)

| Organs/ | Severity of Lesions in Individual Animals | | | | | |
|---|---|---|---|---|---|---|
| Histopathology | I072/019 | I072/020 | I072/021 | I072/022 | I072/023 | I072/024 |
| RIGHT EYE | W | W | W | W | W | W |
| LEFT EYE | | | | | | |
| LENS | | | | | | |
| Liquefaction | 1+ | 1+ | 2+ | 2+ | W | W |
| Epithelial | 2+ | 1+ | 1+ | 1+ | W | 1+ |
| Proliferation | | | | | | |
| CORNEA | — | — | — | — | — | — |
| Corneal edema | 2+ | 1+ | 1+ | 2+ | 1+ | 1+ |

TABLE 8
Main Group/Formulation: G5/F4 (I072/025-I072/030)

| Organs/ | Severity of Lesions in Individual Animals | | | | | |
|---|---|---|---|---|---|---|
| Histopathology | I072/025 | I072/026 | I072/027 | I072/028 | I072/029 | I072/030 |
| RIGHT EYE | W | W | W | W | W | W |
| LEFT EYE | | | | | | |
| LENS | | | | | | |
| Liquefaction | 1+ | W | 1+ | W | W | W |
| Epithelial | W | W | 1+ | W | W | W |
| Proliferation | | | | | | |
| CORNEA | — | — | — | — | — | — |
| Corneal edema | W | W | W | 1+ | W | W |

Right Eye Uninduced/untreated; Left Eye- Induced/treated
Key: W = within normal histological limits, Severity of lesions- 1+ - minimal, 2+ - mild, 3+ - moderate, 4+ -marked, 5+ - Severe.

TABLE 9
Intraocular Pressure: Gender: MaleNo. of Animals/Group:

| | G1 | | | | G2 | | | | G3 | | | |
|---|---|---|---|---|---|---|---|---|---|---|---|---|
| | Left | | Right | | Left | | Right | | Left | | Right | |
| Day | Mean | SD | Mean | SD | Mean | SD | Mean | SD | Mean | SD | Mean | SD |
| 3 | 52.33 | 6.01 | 18.1 | 2.07 | 54.94 | 2.80 | 18.11 | 1.86 | 53.11 | 3.64 | 17.44 | 2.46 |
| 6 | 46.83 | 2.14 | 19.8 | 1.49 | 54.17↑ | 9.78 | 19.44 | 1.57 | 56.22↑ | 5.92 | 19.44 | 0.98 |
| 9 | 53.22 | 5.78 | 20.0 | 1.04 | 57.50 | 5.01 | 19.28 | 2.59 | 54.56 | 5.73 | 19.67 | 0.67 |
| 12 | 49.61 | 4.99 | 18.7 | 2.37 | 40.22↓↓ | 3.75 | 18.83 | 2.87 | 36.61↓↓ | 1.27 | 19.50 | 2.00 |
| 15 | 52.72 | 5.24 | 20.1 | 2.21 | 39.72↓↓ | 5.24 | 19.17 | 1.67 | 39.00↓↓ | 3.74 | 19.72 | 2.07 |
| 18 | 51.44 | 5.94 | 19.7 | 1.79 | 47.22 | 5.92 | 20.22 | 1.61 | 46.22 | 3.26 | 20.06 | 1.90 |
| 21 | 50.39 | 3.92 | 18.9 | 2.71 | 48.50 | 7.27 | 19.17 | 0.62 | 44.67 | 7.42 | 20.00 | 1.51 |
| 24 | 48.56 | 5.58 | 20.2 | 2.41 | 47.89 | 8.83 | 19.39 | 1.22 | 35.11↓↓ | 5.30 | 20.11 | 1.70 |
| 27 | 49.59 | 7.65 | 19.8 | 1.28 | 45.44 | 4.51 | 20.11 | 1.26 | 38.78↓↓ | 3.81 | 20.17 | 0.94 |

| G4 | | G5 | |
|---|---|---|---|
| Left | Right | Left | Right |

| Day | Mean | SD | Mean | SD | Mean | SD | Mean | SD |
|---|---|---|---|---|---|---|---|---|
| 3 | 51.61 | 1.93 | 20.28 | 2.92 | 49.17 | 2.70 | 19.11 | 1.44 |
| 6 | 60.11↑↑ | 4.25 | 21.72 | 12.39 | 59.72↑↑ | 2.68 | 19.83 | 1.07 |
| 9 | 56.00 | 6.93 | 16.94 | 1.82 | 55.78 | 2.15 | 19.83 | 0.84 |
| 12 | 31.33↓↓ | 3.76 | 20.78 | 1.05 | 29.67↓↓ | 2.84 | 18.67 | 1.38 |
| 15 | 31.28↓↓ | 2.83 | 18.89 | 2.36 | 26.00↓↓ | 3.53 | 19.17 | 3.07 |
| 18 | 24.11↓↓ | 3.19 | 19.33 | 1.96 | 20.06↓↓ | 1.94 | 19.33 | 1.52 |
| 21 | 24.28↓↓ | 1.27 | 17.50 | 1.41 | 19.17↓↓ | 2.52 | 20.22 | 1.78 |
| 24 | 22.56↓↓ | 2.79 | 19.61 | 1.48 | 20.33↓↓ | 1.66 | 19.39 | 1.83 |
| 27 | 26.56↓↓ | 3.08 | 20.00 | 1.94 | 20.06↓↓ | 0.98 | 20.56 | 1.76 |

NIL = No Statistical Significance, ↑ = Significantly high at P ≤ 0.05, ↓ = Significantly low at P ≤ 0.05, ↓↓ = Significantly low at P ≤ 0.01, ↑↑ = Significantly high at P ≤ 0.01 Values are mean and % difference in means as compared to respective control

TABLE 10
Formulation details:

| | Formulation number | | | | |
|---|---|---|---|---|---|
| | F1 | F2 | F3 | F4 | F5 |
| | Group | | | | |
| | G2 | G3 | G4 | G5 | G6 |
| | Cinnamic acid derivative | | | | |
| | | Encapsulated | Caffeic Acid | Caffeic Acid | Caf- |
| | Rosmarinic | Rosmarinic | Phenyl ethyl | Phenylethyl | feic |
| | acid | acid | Ester | Amide | acid |
| Concen- | 0.5 | 0.5 | 0.5 | 0.5 | 0.25 |
| tration | | | | | |

TABLE 11
Macroscopic Examination:

| | | | | | | | |
|---|---|---|---|---|---|---|---|
| Gross findings | G1 | G2 | G3 | G4 | G5 | G6 | G7 |

Right Eye (Uninduced)

| | | | | | | | |
|---|---|---|---|---|---|---|---|
| No Abnormality | 6/6 | 6/6 | 6/6 | 6/6 | 6/6 | 6/6 | 6/6 |
| Detected | | | | | | | |

Left eye (Cataract Induced)

| | | | | | | | |
|---|---|---|---|---|---|---|---|
| No Abnormality | 0/6 | 1/6 | 0/6 | 0/6 | 0/6 | 1/6 | 2/6 |
| Detected | | | | | | | |
| Decreased size, | 1/6 | — | 1/6 | — | — | — | — |
| eyeball | | | | | | | |

Opacity, cornea and red discoloured

| | | | | | | | |
|---|---|---|---|---|---|---|---|
| With diffuse opacity | | | | | | | |
| Minimal | — | 1/6 | — | — | — | — | — |
| Mild | — | — | 1/6 | — | — | — | — |
| Moderate | 2/6 | — | — | 4/6 | 1/6 | 1/6 | 4/6 |

Opacity, cornea and red discoloured

| | | | | | | | |
|---|---|---|---|---|---|---|---|
| Opacity, cornea, | | | | | | | |
| diffuse | | | | | | | |
| Minimal | — | 3/6 | — | 1/6 | 3/6 | 1/6 | — |
| Mild | — | 1/6 | 3/6 | — | 2/6 | 1/6 | — |
| Moderate | 4/6 | — | — | 1/6 | — | 1/6 | — |
| Opacity, cornea, focal | — | — | — | — | — | — | — |

TABLE 12
Microscopic Observations (Histopathology):

| | | | | | | | |
|---|---|---|---|---|---|---|---|
| Histopathology findings | G1 | G2 | G3 | G4 | G5 | G6 | G7 |

Right Eye- Non-induced

| | | | | | | | |
|---|---|---|---|---|---|---|---|
| Within Normal Histological | 6/6 | 6/6 | 6/6 | 6/6 | 6/6 | 6/6 | 6/6 |
| Limits | | | | | | | |

Left eye- (Cataract Induced)

| | | | | | | | |
|---|---|---|---|---|---|---|---|
| Within Normal Histological | 0/6 | 0/6 | 1/6 | 0/6 | 0/6 | 0/6 | 2/6 |
| Limits | | | | | | | |

LENS

| | | | | | | | |
|---|---|---|---|---|---|---|---|
| Lenticular Degeneration | 6/6 | 4/6 | 5/6 | 6/6 | 5/6 | 6/6 | 4/6 |
| Epithelial proliferation | 6/6 | 2/6 | 3/6 | 6/6 | 2/6 | 3/6 | 4/6 |
| Absent | — | 4/6 | 3/6 | — | 4/6 | 3/6 | 2/6 |
| Minimal | — | 1 | 1 | 2 | — | 1 | — |
| Mild | — | — | 2 | 3 | 2 | 1 | — |
| Moderate | 3 | — | — | — | — | 1 | 2 |
| Marked | 1 | 1 | — | 1 | — | — | 2 |
| Severe | 2 | — | — | — | — | — | — |
| Liquefaction | 6/6 | 4/6 | 5/6 | 6/6 | 5/6 | 4/6 | 4/6 |
| Absent | — | 2/6 | 1/6 | — | 1/6 | 2/6 | 2/6 |
| Minimal | — | 3 | 3 | — | 3 | 2 | — |
| Mild | 1 | — | 2 | 2 | 2 | 1 | 1 |
| Moderate | 5 | — | — | 4 | — | 1 | 3 |
| Marked | — | 1 | | — | — | — | — |
| Severe | — | — | — | — | — | — | — |

### Intraocular Pressure

Intra-ocular pressures were measured in animals in each group once in three days using a Tonometer. The readings were documented for each eye separately. Intraocular pressure (IOP) was revealed significant decrease (P≤0.01) on Day 36 in all the groups animals treated with cinnamic acid derivatives.

TABLE 13
Gender: Male, No. of Animals/Group: 6

| | G1 | | | | G2 | | | | G3 | | | | G4 | | | |
|---|---|---|---|---|---|---|---|---|---|---|---|---|---|---|---|---|
| | Left | | Right | | Left | | Right | | Left | | Right | | Left | | Right | |
| Day | Mean | SD | Mean | SD | Mean | SD | Mean | SD | Mean | SD | Mean | SD | Mean | SD | Mean | SD |
| 3 | 76.05 | 7.31 | 20.48 | 0.85 | 83.15 | 2.62 | 20.22 | 1.36 | 89.00 | 3.71 | 22.73 | 3.05 | 88.07 | 4.61 | 24.12 | 3.27 |
| 6 | 91.05 | 7.41 | 21.17 | 3.66 | 77.55 | 11.85 | 15.95 | 2.74 | 73.55 | 14.81 | 21.22 | 2.99 | 84.95 | 4.22 | 23.73 | 3.82 |
| 9 | 81.33 | 14.67 | 19.23 | 2.76 | 78.72 | 11.98 | 17.72 | 2.06 | 73.57 | 14.82 | 22.72 | 1.67 | 84.67 | 4.26 | 25.85 | 3.83 |
| 12 | 82.72 | 13.81 | 19.15 | 2.57 | 66.83 | 17.83 | 19.57 | 1.62 | 65.60 | 17.02 | 19.67 | 1.07 | 55.95 | 3.23 | 16.68 | 2.90 |
| 15 | 80.22 | 6.46 | 19.07 | 2.36 | 48.83 | 3.44 | 19.38 | 2.37 | 52.67 | 5.22 | 20.52 | 2.41 | 42.80 | 2.19 | 19.17 | 1.90 |
| 18 | 83.77 | 7.32 | 18.33 | 1.84 | 48.48 | 3.69 | 19.12 | 2.33 | 51.05 | 6.54 | 19.43 | 1.91 | 42.18 | 3.84 | 19.27 | 3.02 |
| 21 | 83.27 | 8.14 | 17.17 | 2.62 | 46.77 | 4.05 | 18.00 | 2.40 | 48.90 | 6.64 | 18.33 | 2.50 | 40.93 | 3.56 | 18.38 | 2.70 |
| 24 | 84.92 | 4.76 | 16.95 | 3.02 | 44.55 | 3.24 | 17.83 | 2.10 | 45.28 | 5.08 | 17.38 | 2.24 | 39.67 | 3.34 | 17.23 | 1.60 |
| 27 | 84.62 | 5.72 | 17.83 | 3.22 | 42.62 | 6.55 | 18.17 | 2.23 | 44.22 | 4.86 | 17.62 | 1.74 | 36.27 | 4.83 | 17.00 | 1.62 |
| 30 | 82.62 | 7.36 | 17.67 | 3.30 | 23.27 | 6.47 | 16.00 | 2.76 | 26.28 | 11.02 | 14.78 | 1.94 | 39.42 | 7.40 | 21.12 | 1.36 |
| 33 | 80.88 | 5.99 | 17.05 | 3.16 | 18.45↓↓ | 2.93 | 17.35 | 2.36 | 30.08↓↓ | 9.50 | 19.57 | 2.95 | 41.85↓↓ | 6.73 | 21.65 | 1.32 |

| | G5 | | | | G6 | | | | G7 | | | |
|---|---|---|---|---|---|---|---|---|---|---|---|---|
| | Left | | Right | | Left | | Right | | Left | | Right | |
| Day | Mean | SD | Mean | SD | Mean | SD | Mean | SD | Mean | SD | Mean | SD |
| 3 | 86.65 | 2.25 | 26.28 | 4.53 | 90.35 | 2.07 | 19.12 | 1.78 | 91.05 | 2.48 | 20.00 | 3.07 |
| 6 | 79.88 | 16.62 | 19.45 | 2.44 | 63.22 | 10.72 | 23.78 | 6.25 | 69.27 | 3.98 | 20.10 | 1.03 |
| 9 | 78.77 | 17.81 | 19.27 | 2.49 | 62.17 | 11.14 | 24.78 | 7.29 | 67.55 | 3.66 | 25.22 | 5.49 |
| 12 | 72.72 | 9.96 | 18.72 | 2.01 | 65.93 | 9.61 | 15.17 | 2.41 | 58.50 | 15.60 | 20.17 | 1.17 |
| 15 | 50.78 | 2.61 | 18.55 | 3.00 | 45.45 | 3.70 | 20.05 | 2.78 | 57.33 | 6.32 | 18.83 | 1.73 |
| 18 | 49.95 | 3.34 | 18.33 | 3.08 | 45.73 | 3.94 | 16.35 | 3.19 | 56.17 | 4.79 | 18.83 | 1.72 |
| 21 | 45.67 | 3.56 | 16.98 | 2.43 | 41.50 | 3.94 | 15.45 | 1.42 | 56.28 | 3.64 | 32.33 | 32.03 |
| 24 | 49.77 | 6.07 | 15.80 | 2.38 | 38.93 | 2.81 | 14.78 | 0.94 | 51.77 | 6.99 | 18.28 | 1.25 |
| 27 | 36.27 | 3.86 | 16.72 | 1.82 | 32.12 | 4.15 | 17.95 | 2.16 | 50.32 | 6.16 | 16.38 | 1.47 |
| 30 | 25.85 | 7.69 | 18.45 | 2.41 | 30.78 | 10.59 | 15.53 | 3.71 | 48.38 | 3.64 | 17.55 | 1.04 |
| 33 | 36.68↓↓ | 5.86 | 19.50 | 2.17 | 26.60↓↓ | 6.05 | 18.17 | 3.63 | 43.63 | 2.12 | 21.07 | 1.67 |

NIL = No Statistical Significance, ↑ = Significantly high at P ≤ 0.05, ↓ = Significantly low at P ≤ 0.05, ↓↓ = Significantly low at P ≤ 0.01, ↑↑ = Significantly high at P ≤ 0.01 Values are mean and % difference in means as compared to respective control.

TABLE 14
Formulation details:

| Group | G2 | G3 | G4 | G5 | G6 | G1 |
|---|---|---|---|---|---|---|
| Cinnamic | Rosmarinic acid + | Caffeic | Epigallocatechin | Encapsulated | Ferulic | Placebo |
| acid | Caffeic acid | acid | Gallate | feruloyl | acid | |
| derivative | | | | Tyramine | | |
| Concentration | 0.5 + 0.25% | 0.5 | 0.5 | 0.5 | 0.5 | 0 |

TABLE 15
Macroscopic Examination:

| Gross findings | G1 | G2 | G3 | G4 | G5 | G6 |
|---|---|---|---|---|---|---|
| Right Eye (Un-induced) | | | | | | |
| No Abnormality Detected | 6/6 | 6/6 | 6/6 | 6/6 | 6/6 | 6/6 |
| Left eye (Cataract Induced) | | | | | | |
| No Abnormality Detected | 0/6 | 4/6 | 6/6 | 6/6 | 6/6 | 6/6 |
| Opacity, cornea, diffuse | | | | | | |
| Minimal | — | 2/6 | — | — | — | — |
| Mild | 3/6 | — | — | — | — | — |
| Moderate | 3/6 | — | — | — | | — |

TABLE 16
Microscopic Examination (Histopathology):

| Histopathology findings | G1 | G2 | G3 | G4 | G5 | G6 |
|---|---|---|---|---|---|---|
| Right Eye- Non-induced | | | | | | |
| No abnormality | 6/6 | 6/6 | 6/6 | 6/6 | 6/6 | 6/6 |
| Left eye- Induced | | | | | | |
| No abnormality | 0/6 | 1/6 | 1/6 | — | 1/6 | — |
| LENS | | | | | | |
| Liquefaction | | | | | | |
| Minimal | — | 2/6 | 5/6 | — | 2/6 | 1/6 |
| Mild | 2/6 | — | — | 6/6 | 1/6 | 4/6 |
| Moderate | — | 2/6 | — | — | 1/6 | 1/6 |
| Marked | — | — | — | — | — | — |
| Severe | 4/6 | 1/5 | — | — | 1/5 | — |
| Epithelial proliferation | | | | | | |
| Severe | 4/6 | — | — | — | 1/5 | — |
| CORNEAP | | | | | | |
| Oedema | | | | | | |
| Mild | — | 1/6 | — | — | — | — |
| Inflammation | | | | | | |
| Minimal | 1/6 | — | — | — | — | — |
| Mild | — | — | — | 1/6 | — | — |
| Moderate | 1/6 | — | — | — | — | — |

TABLE 17
Intraocular Pressure: Gender: Male \No. of Animals/Group: 6

| | G1 | | | | G2 | | | | G3 | | | |
|---|---|---|---|---|---|---|---|---|---|---|---|---|
| | Left | | Right | | Left | | Right | | Left | | Right | |
| Day | Mean | SD | Mean | SD | Mean | SD | Mean | SD | Mean | SD | Mean | SD |
| 3 | 73.22 | 9.47 | 16.83 | 1.20 | 57.56 | 11.4 | 16.44 | 1.59 | 41.50 | 9.37 | 15.83 | 1.51 |
| 6 | 49.22 | 2.90 | 19.00 | 2.54 | 30.50 | 5.23 | 18.06 | 2.39 | 23.89 | 5.89 | 19.11 | 2.66 |
| 9 | 65.00 | 8.87 | 17.17 | 0.80 | 30.33 | 2.93 | 17.06 | 0.88 | 21.61 | 1.51 | 16.61 | 1.87 |
| 12 | 78.83 | 8.14 | 16.33 | 1.04 | 44.11 | 2.87 | 18.00 | 1.61 | 25.33 | 3.86 | 16.94 | 1.32 |
| 15 | 72.94 | 8.22 | 14.17 | 2.26 | 33.50 | 4.71 | 15.28 | 2.88 | 25.11 | 4.16 | 14.61 | 2.46 |
| 18 | 72.67 | 6.46 | 14.00 | 1.81 | 40.17 | 3.64 | 13.56 | 2.46 | 29.56 | 2.97 | 15.44 | 1.47 |
| 21 | 83.00 | 7.47 | 16.61 | 2.10 | 34.06 | 2.64 | 15.50 | 2.39 | 29.94 | 2.41 | 15.28 | 1.79 |
| 25 | 70.89 | 3.50 | 16.78 | 1.12 | 40.56 | 3.91 | 16.56 | 1.97 | 24.78 | 2.48 | 17.17 | 1.88 |
| 28 | 57.00 | 7.61 | 16.78 | 1.60 | 23.22↓↓ | 9.06 | 15.89 | 2.50 | 21.11↓↓ | 5.64 | 17.11 | 1.37 |

| | G4 | | | | G5 | | | | G6 | | | |
|---|---|---|---|---|---|---|---|---|---|---|---|---|
| | Left | | Right | | Left | | Right | | Left | | Right | |
| Day | Mean | SD | Mean | SD | Mean | SD | Mean | SD | Mean | SD | Mean | SD |
| 3 | 29.72 | 2.77 | 18.11 | 2.18 | 50.33 | 9.92 | 18.39 | 1.93 | 30.39 | 2.87 | 17.28 | 1.85 |
| 6 | 33.06 | 3.85 | 16.94 | 2.29 | 33.28 | 2.78 | 18.22 | 2.27 | 28.39 | 6.60 | 16.89 | 1.61 |
| 9 | 26.56 | 2.59 | 16.78 | 1.01 | 31.28 | 2.33 | 16.17 | 1.54 | 28.28 | 3.32 | 16.89 | 0.99 |
| 12 | 32.50 | 4.92 | 17.00 | 1.06 | 30.00 | 3.82 | 17.06 | 1.62 | 25.33 | 3.58 | 17.56 | 1.64 |
| 15 | 26.39 | 4.87 | 12.56 | 3.64 | 28.17 | 3.03 | 13.11 | 1.97 | 26.00 | 2.69 | 14.94 | 2.35 |
| 18 | 28.94 | 2.61 | 16.61 | 1.44 | 29.78 | 2.52 | 14.72 | 1.52 | 26.56 | 1.96 | 14.94 | 2.03 |
| 21 | 27.11 | 2.97 | 16.06 | 1.62 | 28.06 | 2.48 | 17.56 | 2.87 | 23.22 | 2.83 | 17.06 | 1.21 |
| 25 | 25.56 | 1.76 | 18.17 | 2.50 | 25.39 | 1.54 | 18.06 | 1.82 | 21.72 | 1.47 | 17.61 | 2.15 |
| 28 | 20.00↓↓ | 2.05 | 16.11 | 2.04 | 23.33↓↓ | 3.54 | 17.28 | 1.60 | 23.67↓↓ | 4.96 | 17.28 | 1.60 |

NIL = No Statistical Significance, ↑ = Significantly high at P ≤ 0.05, ↓ = Significantly low at P ≤ 0.05, ↓↓ = Significantly low at P ≤ 0.01, ↑↑ = Significantly high at P ≤ 0.01 Values are mean and % difference in means as compared to respective control

### Detailed Description of Efficacy Studies:

Macroscopic examination revealed that there were no abnormalities found in all the 6 animals treated 0.5% Rosmarinic acid (Table 3, G5 group) Microscopic examination revealed that in four animals, liquefaction in the lens is within histological limits, while in two animals, minimal liquefaction was observed. In five animals' epithelial proliferation in lens was within histological limits, indicating positive effects. Only one animal displayed minimal epithelial proliferation in this context (Table 8, G5 group)

Additionally, when assessing the corneas, it was found that five animals' corneal edema was within histological limits and one animal with minimal corneal edema (Table 8, G5 group)

In a glaucoma-induced left eye, the intraocular pressure initially measured at 59.72 mmHg within six days, while the uninduced right eye maintained a normal intraocular pressure of 19.11 mmHg (Table 9 G5 group). This is in sharp contrast to the untreated placebo group where the intraocular eye pressure remains the same. Following a 28-day treatment regimen with 0.5% Rosmarinic acid, the intraocular pressure in the induced left eye dramatically decreased from 59.72 mmHg to 20.06 mmHg (Table 9 G5 group). This remarkable reduction in intraocular pressure demonstrates the efficacy of Rosmarinic acid in controlling Intraocular Pressure and preventing glaucoma.

Rosmarinic acid (0.5%) as active ingredient demonstrates its efficacy not only in preventing lens liquefaction, epithelial proliferation, and corneal edema and treating cataract but also in reducing intraocular pressure.

Further efficacy studies involve investigating the efficacy of Rosmarinic acid, Caffeic acid, Caffeic Acid Phenylethyl Ester (CAPE), Caffeic Acid Phenylethyl Amide (CAPA), Caffeoyl tyramine, a combination of Rosmarinic and Caffeic acid, Epigallocatechin gallate, Encapsulated feruloyl tyramine, Ferulic acid.

Out of which 0.5% Rosmarinic acid in G2 group and 0.25% caffeic acid in G6 were shown to be the best. In the G2 group, macroscopic examination revealed minimal corneal opacity in some animals, with no abnormalities in one. (Table 11, G2) Histopathological findings showed that all animals were within histological limits. However, lens studies indicated minimal lenticular degeneration in four animals and varying levels of epithelial proliferation and liquefaction (Table 12, G2).

In the G6 group, animals treated with 0.25% caffeic acid exhibited similar results corneal opacity, but a higher incidence of lenticular degeneration (Table 12, G6). The study concluded that Rosmarinic acid at 0.5% demonstrated superior efficacy, while Caffeic acid at 0.25% also showed promise, prompting plans to increase its concentration in further studies.

Caffeic Acid (G3 group): This formulation showed the best results, with remarkable efficacy not only in cataract reduction but also in addressing glaucoma.

Cataract Efficacy: Macroscopic observation revealed that all 6 animals were completely recovered, and no cataract was observed at all (Table 15, G3). Histopathology showed that the cells in the lens, which had degenerated due to cataract induction, were fully regenerated in 5 out of 6 animals, from severe degeneration to a minimal scale. One animal exhibited complete recovery, with the eye appearing normal, similar to an uninduced right eye (Table 16, G3).

IOP and Glaucoma Efficacy: In an IOP-induced left eye, the mean intraocular pressure of six animals was initially measured at a high 41.5 mmHg within three days, while the uninduced right eye maintained a normal intraocular pressure of 16.83 mmHg in contrast to the IOP of untreated placebo group where the IOP initial was 73.22 and final 57.00 mmHg. However, following a 28-day treatment regimen with 0.5% caffeic acid, the intraocular pressure in the induced left eye dramatically decreased from 41.50 mmHg to 21.11 mmHg. This remarkable reduction in intraocular pressure demonstrates the remarkable efficacy of caffeic acid in directly treating glaucoma (Table 17, G3).

In summary, Caffeic Acid (G3) not only excelled in reducing cataracts but also displayed a significant ability in reducing IOP and thereby preventing glaucoma, making it a promising candidate for both ocular conditions.

Epigallocatechin Gallate (G4 group): This formulation exhibited outstanding efficacy in reducing cataracts and also demonstrated a significant capability to address glaucoma.

Cataract Efficacy: Macroscopic observations revealed complete recovery in all 6 animals, with no observable cataracts (Table 15, G4). Histopathological findings indicated that lens cells, initially affected by cataract induction, were fully restored, or regenerated in all 6 out of 6 animals, transitioning from severe degeneration to a milder state. Only one animal exhibited mild corneal inflammation, which is a relatively minor issue given the overall reduction in cataract severity (Table 16, G4).

IOP and Glaucoma Efficacy: In glaucoma-induced left eyes, the mean intraocular pressure of six animals was initially measured at a high 29.72 mmHg within three days, while the uninduced right eyes maintained a normal intraocular pressure of 18.11 mmHg. However, after a 28-day treatment regimen with 0.5% Epigallocatechin Gallate, the intraocular pressure in the induced left eyes dramatically decreased from 29.72 mmHg to 20.00 mmHg.

This remarkable reduction in intraocular pressure demonstrates the impressive efficacy of Epigallocatechin Gallate in directly treating glaucoma (Table 17, G4).

In summary, Epigallocatechin Gallate not only excelled in reducing cataracts but also exhibited a significant capacity to address glaucoma, making it a promising candidate for both conditions.

Encapsulated Feruloyl Tyramine (G5 group): This formulation displayed notable effectiveness in reducing cataracts and demonstrated a remarkable efficacy to address glaucoma.

Cataract Efficacy: Upon macroscopic examination, complete recovery was observed in all 6 animals, with no cataracts detected (Table15, G5). Histopathological analysis indicated that this formulation effectively regenerated lens cells, transitioning them from severe degeneration to a moderate scale in four out of six animals. Additionally, epithelial proliferation was observed in one animal, suggesting a moderate level of efficacy in cataract reduction (Table 16, G5).

IOP and Glaucoma Efficacy: In glaucoma-induced left eyes, the initial mean intraocular pressure of six animals was remarkably high at 50.33 mmHg within three days, while the uninduced right eyes maintained a normal intraocular pressure of 18.39 mmHg. However, following a 28-day treatment regimen with 0.5% Encapsulated Feruloyl Tyramine, the intraocular pressure in the induced left eyes dramatically decreased from 50.33 mmHg to 23.33 mmHg. This substantial reduction in intraocular pressure demonstrates the impressive efficacy of Encapsulated Feruloyl Tyramine in directly treating glaucoma (Table 17, G5).

In summary, encapsulated Feruloyl Tyramine (G5) not only effectively reduced cataracts but also exhibited significant potential in the treatment of glaucoma, making it a promising candidate for both conditions.

Ferulic Acid (G6 group): This formulation led to complete recovery from cataracts in all 6 animals and resulted in minimal to moderate lenticular degeneration, indicating a moderate level of cataract reduction.

IOP, Glaucoma Efficacy: In glaucoma-induced left eyes, the initial mean intraocular pressure was remarkably high at 30.39 mmHg within three days, while the uninduced right eyes maintained a normal intraocular pressure of 17.28 mmHg. However, following a 28-day treatment regimen with 0.5% Ferulic Acid, the intraocular pressure in the induced left eyes dramatically decreased from 30.39 mmHg to 23.67 mmHg, demonstrating impressive efficacy in treating glaucoma (Table 17, G6).

Rosmarinic Acid+Caffeic Acid (G2 group): Four out of six animals experienced complete recovery from cataracts (Table 15)., but variable results were observed, with some animals displaying minimal to severe lenticular degeneration and corneal edema, suggesting relatively lower efficacy. (Table 16, G2).

Glaucoma Efficacy: In glaucoma-induced left eyes, the initial mean intraocular pressure was remarkably high at 57.56 mmHg within three days, while the uninduced right eyes maintained a normal intraocular pressure of 16.44 mmHg. Following a 28-day treatment regimen with 0.5% Rosmarinic Acid+Caffeic Acid, the intraocular pressure in the induced left eyes dramatically decreased from 57.56 mmHg to 23.22 mmHg, indicating impressive efficacy in treating glaucoma (Table 17, G2).

So, the order of efficacy in terms of cataract reduction and reducing intraocular pressure from highest to lowest would be Caffeic Acid G3>Epigallocatechin Gallate G4>Encapsulated Feruloyl Tyramine G5>Ferulic Acid G6>Rosmarinic Acid+Caffeic Acid G2.

The formulations of the present invention achieved complete cataract recovery and a remarkable reduction in intraocular pressure in glaucoma-induced eyes, with complimentary properties.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. An ophthalmic formulation comprising
i. Cinnamic acids or its derivatives as active ingredients; and/or
ii. Catechins or its derivatives as active ingredients.

2. The ophthalmic formulation as claimed in claim 1, wherein the concentration of each of the active ingredient/s ranges between 0.01% to 5.0% w/w.

3. The ophthalmic formulation as claimed in claim 1, wherein the formulation comprises acceptable excipients.

4. The ophthalmic formulation as claimed in claim 1, wherein said formulation is topical and/or injectable and/or oral.

5. The ophthalmic formulation as claimed in claim 1, wherein the formulation is in the form of powder, liquid, gel and the like.

6. The ophthalmic formulation as claimed in claim 1, wherein the cinnamic acids derivatives are either encapsulated or free.

7. The ophthalmic formulation as claimed in claim 1, wherein the cinnamic acids include but not limited to cinnamic acid, coumaric acid, caffeic acid, rosmarinic acid, ferulic acid and their combinations thereof.

8. The ophthalmic formulation as claimed in claim 1, wherein cinnamic acid ester derivatives comprise the esters of general formula (I);
CA-CO—OR (I)
wherein CA=cinnamic acid and R═C1-C22 alkyl, aryl, heteroaryl,
Ar—CH₂—CH₂—, carbohydrates, lipids, and CA-CO—O(CH)nO—CO-CA
wherein n=2-10.

9. The ophthalmic formulation as claimed in claim 8, wherein cinnamic acid esters include but not limited to ethyl cinnamate, ethyl p-methoxy cinnamate, butyl cinnamate, amyl cinnamate, benzyl cinnamate, phenethyl cinnamate, tyrosol cinnamate, hydroxy tyrosol cinnamate, isobutyl cinnamate, cinnamyl cinnamate, phenethyl coumarate, tyrosol coumarate, hydroxy tyrosol coumarate, n-hexadecyl coumarate, n-octadecyl coumarate, n-docosyl coumarate, phenethyl ferulate, tyrosol ferulate, hydroxy tyrosol ferulate, n-hexadecyl ferulate, n-octadecyl ferulate, n-docosyl ferulate, caffeic acid phenylethyl ester (CAPE), tyrosol caffeate, hydroxy tyrosol caffeate, n-hexadecyl caffeate, n-octadecyl caffeate, n-docosyl caffeate, rosmarinic acid, cinnamic acid carbohydrate or lipid esters and their combinations thereof.

10. The ophthalmic formulation as claimed in claim 1, wherein cinnamic acids derivatives comprise the amides of general formula (II);
CA-CO—NHR (II)
wherein CA=cinnamic acid and R is C1-C22 alkyl, aryl, heteroaryl, Aralkyl, lipids and carbohydrates.

11. The ophthalmic formulation as claimed in claim 10, wherein cinnamic acids amides include but not limited to cinnamoyl phenethyl amine, cinnamoyl tyramine, cinnamoyl serotonin, cinnamoyl dopamine, coumaroyl phenethyl amine, coumaroyl tyramine, coumaroyl dopamine, coumaroyl serotonin, feruloyl phenethyl amine, feruloyl tyramine, feruloyl serotonin, feruloyl dopamine, caffeic acid phenylethyl amide (CAPA), caffeoyl tyramine, caffeoyl serotonin, caffeoyl dopamine, cinnamic acid carbohydrate or lipid amides and their combinations thereof.

12. The ophthalmic formulation as claimed in claim 1, wherein the cinnamic acids derivatives include but not limited to cinnamic acid, coumaric acid, caffeic acid, rosmarinic acid, ferulic acid, caffeic acid phenylethyl amide (CAPA), caffeic acid phenylethyl ester (CAPE), feruloyl tyramine, caffeoyl tyramine, caffeoyl dopamine, caffeoyl serotonin, feruloyl dopamine and feruloyl serotonin either alone or combinations thereof.

13. The ophthalmic formulation as claimed in claim 1, wherein the formulation includes catechins or their esters, amides, glycosides, sulfates, phosphates and the like.

14. The ophthalmic formulation as claimed in claim 1, wherein the catechins derivatives include but not limited to catechins or its derivatives are selected from the group consisting of (a) catechin: (2R,3S)-(+)-catechin; (2R,3S)-2-(3,4-dihydroxyphenyl)-3,4-dihydro-2H-chromene-3,5,7-triol; (b) EC: (2R,3S)-(−)-epicatechin; 2-(3,4-Dihydroxyphenyl)-3,4-dihydro-2H-1-benzopyran-3,5,7-triol; (c) ECG: (2R,3R)-(−)-epicatechin gallate; (2R,3R)-2-(3,4-dihydroxyphenyl)-3,4-dihydro-1(2H)-benzopyran-3,5,7-triol 3-(3,4,5-trihydroxy-benzoate); (d) EGC: (2R,3R)-(−)-epigallocatechin; (2R,3R)-2-(3,4,5-trihydroxyphenyl)-3,4-dihydro-1(2H)-benzopyran-3,5,7-triol;(e) GC: (2S,3R-(−)-gallocatechin; (2S,3R)-2-(3,4,5-trihydroxy phenyl)-3,4-dihydro-1(2H)-benzopyran-3,5,7-triol; (f) EGCG: (2R,3R)-(−)-epigallocatechin gallate; (2R,3R)-2-(3,4,5-trihydroxyphenyl)-3,4-dihydro-1(2H)-benzopyran-3,5,7-triol 3-(3,4,5-trihydroxy benzoate); (g) GCG: (2S,3R)-(−)-gallocatechin gallate; (2S,3R)-2-(3,4,5-trihydroxyphenyl)-3,4-dihydro-1(2H)-benzopyran-3,5,7-triol 3-(3,4,5-trihydroxybenzoate).

15. The ophthalmic formulation as claimed in claim 3, wherein the excipients include but not limited to stability enhancers/preservatives, solubility enhancers, buffering agents, surfactants, pH adjusters, viscosity enhancers or modifiers, diluents, encapsulating agents, antioxidants, vitamins, and the like in suitable proportions.

16. The ophthalmic formulation as claimed in claim 15, wherein the excipients include but not limited to 2-Hydroxypropyl-β-Cyclodextrin, Polysorbate surfactants including Polysorbate 80 (Tween 80) and Polysorbate 20 (Tween 20), Ethanol, Benzyl alcohol, Propylene glycol, Polyvinylpyrrolidone (PVP), ethylenediaminetetraacetic acid (EDTA); viscosity enhancers or modifiers selected from Carboxymethyl Cellulose (CMC) or its acceptable salt, Hyaluronic acid or its acceptable salt, Hydroxypropyl Methylcellulose (HPMC), Polyvinyl Alcohol (PVA), Glycerin, Polyethylene Glycol (PEG), Xanthan Gum, Guar Gum, Gellan gum, Carbomers like Polyacrylic Acid; Buffering agents selected from phosphate buffer including sodium phosphate and potassium phosphate; Citrate buffers selected from sodium citrate or citric acid; Borate buffers such as potassium borate; Acetate buffers such as sodium acetate, Tris Buffer like Tris(tris(hydroxymethyl)aminomethane; HEPES Buffer like (4-(2-hydroxyethyl)-1-piperazineethanesulfonic acid; ascorbic acid; sodium metabisulfite; potassium carbonate; antioxidants selected from glutathione and N-acetyl carnosine; vitamins selected from vitamin E or vitamin A in various combinations and proportions.

17. The ophthalmic formulation as claimed in claim 15, wherein the excipients include but not limited to: viscosity enhancer include carboxymethyl cellulose or an acceptable salt, in an amount ranging from 0.05% to 3.0% w/w, preferably in the range of 0.2% to 2.0% w/w; or hyaluronic acid or an acceptable salt ranging from 0.05% to 1.5% w/w, preferably in the range of 0.05 to 0.3% w/w; glutathione/N-acetyl carnosine (NAC) as an antioxidant in an amount of 0.1 to 5% w/w; 2-hydroxypropyl-β-cyclodextrin as encapsulating agent or as a stabilizer in an amount ranging from 0.1% to 15% w/w, preferably 1 to 5% w/w, based on the total weight of the composition; surfactant selected from tween 80 in an amount ranging from 1-10% w/w preferably 2-6% w/w.

18. The ophthalmic formulation as claimed in claim 1 comprising
i. Active Ingredients: 0.01% w/w to 5% w/w of Cinnamic Acids and/or its Derivatives and/or Catechins and/or its Derivatives (wherein the active ingredients are either encapsulated or in free state);
ii. Appropriate Excipients;
iii. Appropriate pH; and
iv. Sterile Water.

19. The ophthalmic formulation as claimed in claim 1 comprising
i. Active Ingredients: 0.1 to 2% w/w w/w of Caffeic acid and/or its ester Rosmarinic acid and/or Ferulic acid and/or Feruloyl Tyramine and/or Caffeic Acid Phenylethyl Ester and/or Caffeic Acid Phenylethyl Amide and/or Epigallocatechin Gallate and/or Epicatechin Gallate and/or Epigallocatechin and like (wherein the active ingredients are either encapsulated or in free state);
ii. Excipients: 0.3% w/w Carboxymethylcellulose sodium; 0.1% w/w of Sodium hyaluronate; 1-5% w/w of 2-hydroxypropyl-β-cyclodextrin; 6% w/w Tween 80, Potassium Borate, Potassium Bicarbonate, with or without Antioxidants and vitamins in appropriate proportion
iii. Appropriate pH between 6.2 to 7.6;
iv. Isotonic Solution; and
i. Sterile Water.

20. The ophthalmic formulation as claimed in claim 1, wherein said formulation is prepared by a process comprising
i. Dissolving the active ingredients comprising Cinnamic Acid/s or its derivatives and/or Catechin/s or its derivatives in sterile water at a appropriate temperature including but not limited to temperature ranging between 25-75 °C;
ii. Adding appropriate excipients to the solution of step (i) and stirring the mixture at appropriate temperature;
iii. Adjusting the pH in the range with the mixture of base and a buffer; and stirring to a clear and homogenous mixture; and
iv. Packing and storing the ophthalmic formulation of step (iii) at an appropriate temperature including temperature between 2 to 40 °C.

21. The ophthalmic formulation as claimed in claim 20, wherein the active ingredients are either encapsulated or in free state.

22. The ophthalmic formulation as claimed in claim 20, wherein the excipients include but not limited to stability enhancers/preservatives, solubility enhancers, buffering agents, surfactants, pH adjusters, viscosity enhancers or modifiers, diluents, encapsulating agents, antioxidants, vitamins, and the like in suitable proportions.

23. A method for treating/clearing/reducing cataractous lens, intraocular pressure, glaucoma, lenticular degeneration, corneal epithelial proliferation/liquefaction/inflammation, comprising administering the ophthalmic formulation of claim 1 to a subject in need thereof.

24. A method for treating/clearing/reducing cataractous lens, intraocular pressure, glaucoma, lenticular degeneration, corneal epithelial proliferation/liquefaction/inflammation and their occurrence/reoccurrence comprising applying the ophthalmic formulation of claim 1 to an eye of a subject in need thereof.
